(19) United States
(12) Patent Application Publication
(10) Pub. No.: US 2002/0128613 A1
(43) Pub. Date: Sep. 12, 2002

(54) Method of treating eye diseases of animals and artificial lacrimal duct used therefor

(76) Inventor: Masanari Nakayama, Nara-city (JP)

Correspondence Address: Masanari Nakayama, 6-1, Minamihukurocho, Nara-city, 630-8342, JP

(21) Appl. No.: 09/866,167

(22) Filed: May 25, 2001

(30) Foreign Application Priority Data
Mar. 12, 2001 (JP) 2001-118891

(51) Int. Cl. A61M 5/00; A61M 35/00

(52) U.S. Cl. 604/294; 604/8

(57) ABSTRACT

With a method of treating eye diseases of animals according to the present invention, a drug solution is injected to the end of an artificial lacrimal duct placed beneath the skin of the head and the neck from the eyelid toward the rear part of the neck, so that the drug solution can be released from the tip of the artificial lacrimal duct opened in the conjunctiva on the backside of the eyelid, in order that the eyelid of animals that behave violently so as to avoid application of eye drops or eyewashing does not have to be opened forcibly. Moreover, the artificial lacrimal duct used in the method of the present invention is provided with a tip fixation means such as a ring or a hook at the tip portion thereof, thereby enabling reliable fixation of the tip of the artificial lacrimal duct on the backside of the conjunctiva.

### BACKGROUND OF THE INVENTION

1. Field of the Invention

The present invention relates to a method of treating eye diseases of animals and an artificial lacrimal duct used therefor. Since there are many animals which dislike application of eye drops or washing eyes and behave boldly, the present invention is for treating eye diseases of animals by releasing a drug solution such as an eye disease remedium, an eyewash or an artificial lacrimal fluid, from an artificial lacrimal duct provided in a conjunctiva on the backside of the eyelid.

2. Description of the Related Art

Many of animals such as dogs and cats have eye diseases such as keretoconjunctivitis sicca, keratitis, conjunctivitis, uveitis, corneal injuries, corneal ulcers, glaucoma or cataract, which should be treated for a long period of time by applying eye drops or washing eyes. In particular, keretoconjunctivitis sicca should be treated by applying an artificial lacrimal fluid for every two or three hours. Otherwise, the animal loses its sight. Therefore, the owner must continue application of eye drops until its death.

Different from human being, however, there are not so many animals that tamely let us apply eye drops or wash eyes. Since animals instinctively try to avoid water droplet jumping into the eyes, it is natural that they hate application of eye drops or washing eyes, which is performed by forcing the eyelid open. Some resist it by behaving as violently as the owner cannot control. Therefore, veterinarians and owners of animals have heretofore had difficulty in applying eye drops or washing eyes.

### OBJECTS AND SUMMARY OF THE INVENTION

It is therefore an object of the present invention to provide a novel method of treating eye diseases of animals and an artificial lacrimal duct used therefor, which can obtain the same effect as that of application of eye drops or eyewashing without opening the eyelid of animals.

In order to achieve the above object, with the method of treating eye diseases of animals of the present invention, a long and slender artificial lacrimal duct is placed beneath the skin of the head and the neck from the eyelid toward the rear part of the neck; and the tip of the artificial lacrimal duct is fixed in the conjunctiva on the backside of the eyelid. Then, a syringe or a pump of a transfusion device is fitted to the end of the artificial lacrimal duct that is opened in the skin at the rear of the neck, to thereby inject a drug solution such as an eye disease remedium, an eyewash or an artificial lacrimal fluid. The drug solution injected to the artificial lacrimal duct is released to the inside of the eyelid, that is, to the conjunctiva or onto the surface of the eyeball. Hence, it serves the same function as that of the conventional application of eye drops or eyewashing.

As described above, by providing the artificial lacrimal duct in the conjunctiva on the backside of the eyelid, eye diseases can be treated without opening the eyelid of animals. Therefore, it is not necessary to hold down animals that behave violently to avoid application of eye drops or eyewashing, nor to open the eyelid forcibly. As a result, it can greatly alleviate a burden imposed on the veterinarians and owners of animals. Moreover, it is easy to let the drug solution flow into the inside of the eyelid as required. Hence, the effect of treatment of eye diseases can be improved remarkably, enabling a great reduction of the period required until being completely cured. As a result, the object to provide a novel method of treating eye diseases of animals, which replaces the conventional application of eye drops or eyewashing, can be realized by very simple means, that is, hypodermic placement of an artificial lacrimal duct.

Moreover, the artificial lacrimal duct used in the method of treating eye diseases of the present invention is provided with, as means for fixing the tip, a ring for suturing to the conjunctiva, or a hook for hooking to the conjunctiva, at the tip of a soft long and slender tube. By having this means, the tip of the artificial lacrimal duct can be reliably fixed to the conjunctiva on the backside of the eyelid. When the tip of the artificial lacrimal duct is clamped between an alligator forceps and inserted through beneath the skin, the ring or the hook serves as a protrusion, and hence it can be reliably clamped by the forceps.

The artificial lacrimal duct is preferably a soft small tube made of silicon or vinyl chloride, but the material thereof is not particularly limited. The artificial lacrimal duct is preferably a single tube having a length of about 30 to 40 cm, and an outer diameter of about 1 to 2 mm. Alternatively, a two-pass catheter used for bladder-washout or the like may be used instead of the single tube. That is to say, one pass is used for releasing the eye disease remedium, and the other is used for flowing a physiological salt solution to wash eyes. Moreover, one pass is fixed in the vicinity of the inner corner of the eye, and the other is fixed in the vicinity of the outer corner of the eye, to thereby suck out the drug solution released into the eyelid from the artificial lacrimal duct at the inner corner of the eye, by a suction pump connected to the artificial lacrimal duct provided at the outer corner of the eye. In this manner, the hair around the eyes can be prevented from becoming dirty due to the drug solution.

The tip fixation means provided in the artificial lacrimal duct is preferably a ring-type protrusion for sewing on the conjunctiva with a suture thread, or a hook-type protrusion for hooking to the conjunctiva for fixation. These tip fixation means are for the purpose that the tip of the artificial lacrimal duct is prevented from protruding from the conjunctiva, and hence, is provided substantially squarely to the tip portion of the artificial lacrimal duct. The number of rings may be one or two. The size of the ring is desirably the same as the outer diameter of the artificial lacrimal duct or smaller. Moreover, these tip fixation means may be integrally formed with the artificial lacrimal duct, or a separately produced fixing means may be bonded to the tip portion of the artificial lacrimal duct.

In order to prevent various bacteria from coming into from the end of the artificial lacrimal duct placed hypodermically, it is desirable that an end sealing means is provided at the end portion. As the simplest end sealing means, a syringe may be left attached always at the end of the artificial lacrimal duct exposed outside of the skin at the rear part of the neck. Alternatively, a rubber cap may be put on over the end of the artificial lacrimal duct exposed outside the skin, and a needle of a syringe may be pierced into the rubber cap to thereby inject the drug solution. Alternatively, a tubular end sealing tool (manufactured by BARD Co., USA, referred to as a reservoir or a boat), with the opening coated with an artificial skin, may be mounted at the end of the artificial lacrimal duct, and laid under the skin, and a needle of a syringe may be pierced into this artificial skin to thereby inject the drug solution into the artificial lacrimal duct.

As the drug solution injection means provided at the end of the artificial lacrimal duct, the most convenient means is a syringe. Also, if the animal is in an animal hospital, an electric pump with a timer for transfusion may be hung from a cage accommodating the animal and connected to the end of the artificial lacrimal duct, so that a certain amount of drug solution can be released to the inside of the eyelid for every several hours, all day long including nighttime. Moreover, when the owner administers it at home, it is convenient to attach a small pump to a collar, to make the animal carry it in a knapsack for animals, to put it on the back of the animal by an adaptic dressing (product name: Press Net), or to sew the small pump to the skin on the back. The type of the small pump is not particularly limited, but for example, INFU-Disk (product name) manufactured by MED-E-CELL Co., USA is suitable.

When the artificial lacrimal duct constituted as described above is inserted to beneath the skin around the head and the neck, the key place of the skin is cut open to make a small hole, and an alligator forceps is inserted to beneath the skin from this small hole. Then, the alligator forceps is forwarded beneath the skin and projected from another small hole, and pulled towards this side, by clamping the tip portion of the artificial lacrimal duct by the alligator at the tip of the forceps. By repeating this procedure several times, the artificial lacrimal duct is guided from one small hole to the next, to thereby bring out the artificial lacrimal duct on the backside of the eyelid.

The position of the above-described small holes are preferably at four points, that is, near the fifth cervical vertebra at the rear of the neck, near the regio parietalis capitis, near the upper part of the eyebrow, and in the conjunctiva exposed by reversing the upper eyelid. Alternatively, the artificial lacrimal duct drawn to the regio parietalis capitis may be pulled down to beneath the skin of the regio buccalis by providing a small hole in the regio buccalis, and guided from there to the conjunctiva on the backside of the lower eyelid.

When the artificial lacrimal duct is provided in both eyes, after two artificial lacrimal ducts are drawn from the rear part of the neck to the small hole near the regio parietalis capitis, one artificial lacrimal duct is guided to the left eye, and the other is guided to the right eye. Alternatively, an artificial lacrimal duct branching out into two like the rubber tube of a stethoscope may be used, so as to be inserted through beneath the skin on the opposite sides of the face from the vicinity of the regio parietalis capitis to the right and left sides, separately.

Different from the human skin, the skin of animals is thick and strong, and beneath the skin into which the artificial lacrimal duct is inserted, important nerves and blood vessels are not distributed. Therefore, even if the alligator forceps inserted to beneath the skin is forwarded forcibly from a small hole to another small hole, any trouble caused in the animal is not observed. Also, since the eyelid of animals is much deeper than the human eyelid, if the tip of the artificial lacrimal duct is fixed in the conjunctiva near the deepest portion, there is no possibility that the tip is brought into contact with the cornea. Hence, the fixed part of the artificial lacrimal duct which, in the case of human, would be brought into contact with the cornea and cause uncomfortable feeling, does not cause any problem with respect to animals.

Moreover, immediately after completion of the operation, by injecting a drug solution mixing antibiotics, atropine, artificial lacrimal fluid and the like to the end of the artificial lacrimal duct, the drug solution can be released into the eyelid that is closed due to anesthesia. Hence, the sutured part of the incised conjunctiva can be sufficiently disinfected and washed. As a result, the tissue in the vicinity of the tip portion of the artificial lacrimal duct fixed in the conjunctiva can be reliably prevented from festering.

### BRIEF DESCRIPTION OF THE DRAWINGS

The features, objects and advantages of the present invention will become more apparent from the detailed description, when taken in conjunction with the following drawings, in which:

FIG. 1 is a diagram showing one embodiment of a method of treating eye diseases according to the present invention;

FIG. 2 is a diagram showing a procedure for inserting an artificial lacrimal duct used in the present invention to beneath the skin;

FIG. 3 is a diagram showing one example of a suitable region to which the tip of the artificial lacrimal duct used in the present invention is fixed;

FIG. 4 is a diagram showing an example of use of a small pump in the method of treating eye diseases according to the present invention;

FIG. 5 is a perspective view showing an embodiment of a tip fixation means provided in the artificial lacrimal duct used in the present invention; and

FIG. 6 is a perspective view showing another embodiment of the tip fixation means provided in the artificial lacrimal duct used in the present invention.

### DETAILED DESCRIPTION OF THE PREFERRED EMBODIMENTS

Preferred embodiments will now be described with reference to the drawings. FIG. 1 is a diagram showing one embodiment of a method of treating eye diseases according to the present invention. In this embodiment, an artificial lacrimal duct 1 is guided to the conjunctiva on the backside of the upper eyelid in the vicinity of the outer corner of the eye of the right eye, and tied up and sutured to the conjunctiva by passing a suture thread 4 through a ring of a tip fixation means 2. At the time of suturing, the edge of the eyelid is clamped by a forceps 5, and dragged to reverse the eyelid, to thereby expose the conjunctiva. The fixing position of the artificial lacrimal duct is not limited to the vicinity of the outer corner of the eye, but may be the central region of the eyelid or the vicinity of the inner corner of the eye.

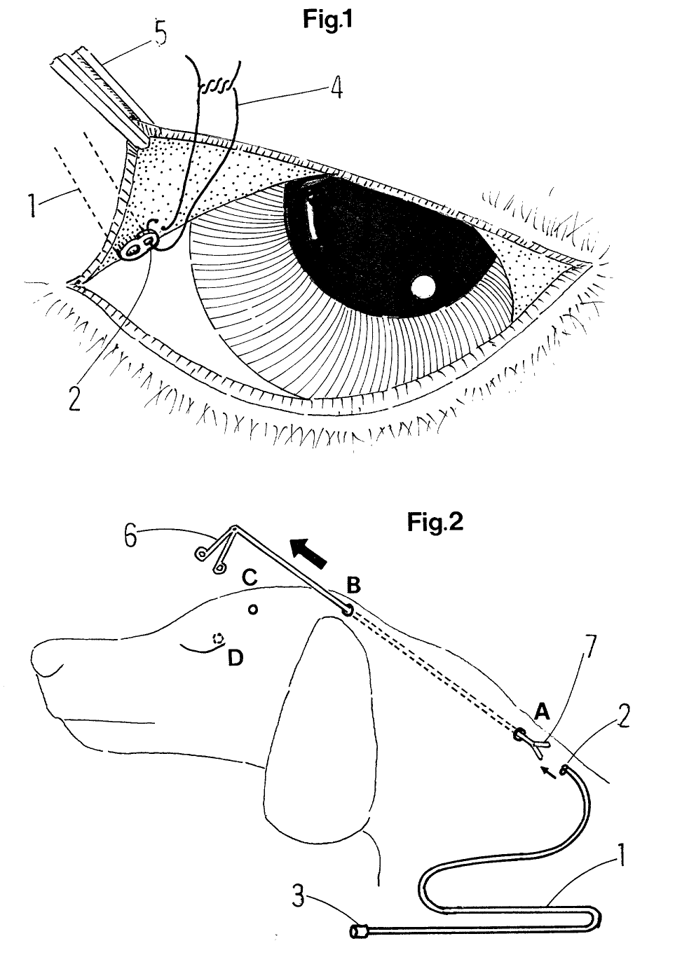

FIG. 2 is a diagram showing a procedure for inserting the artificial lacrimal duct of the present invention to beneath the skin. At first, the hair around the head and the neck of the animal anesthetized over the whole body is shaved to thereby expose the skin. Then, the skin in the vicinity of the fifth cervical vertebra A, the skin in the vicinity of the regio parietalis capitis B, the skin in the vicinity of the upper part of the eyebrow C, and the conjunctiva in the vicinity of the outer corner D of the eye of the upper eyelid are respectively incised by about 5 to 8 mm, to form small holes at four points.

Next, an alligator forceps 6 is inserted into the hole B formed in the skin in the vicinity of the regio parietalis capitis, and forwarded forcibly beneath the skin of the neck toward the hole A formed in the skin at the rear of the neck. Then, the tip of the alligator forceps 6 is projected from the hole A, and the alligator 7 is opened to clamp the tip portion of the artificial lacrimal duct 1 having the tip fixation means 2, and retreated as it is to thereby draw out the tip portion of the artificial lacrimal duct 1 from the hole B.

Subsequently, the alligator forceps 6 inserted into the hole C in the vicinity of the upper part of the eyebrow is forwarded beneath the skin around the head toward the hole B, to clamp the artificial lacrimal duct 1 pulled out from the hole B and draw it to the hole C. Then, the alligator forceps 6 is inserted from the hole D in the conjunctiva exposed by reversing the upper eyelid, to thereby pull out, to the hole D, the tip of the artificial lacrimal duct 1 drawn to the hole C.

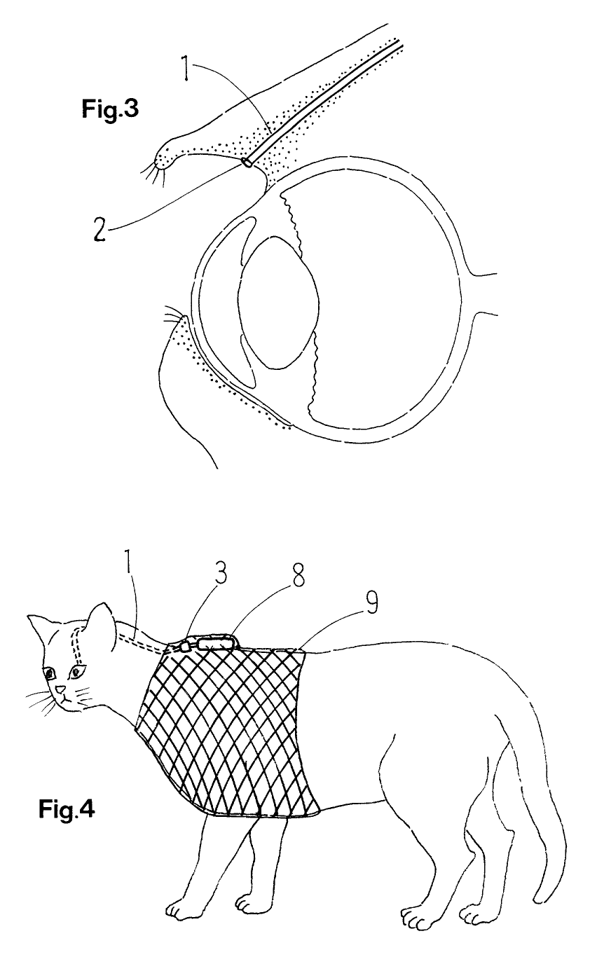

FIG. 3 is a diagram showing one example of a suitable region to which the tip of the artificial lacrimal duct used in the present invention is fixed. The artificial lacrimal duct 1 guided to the backside of the upper eyelid is fixed to the conjunctiva near the deepest part of the eyelid, using the tip fixation means 2. At this time, though not shown, the incised part of the conjunctiva provided for inserting the alligator forceps is also closed by tying up by a suture thread. If the tip of the artificial lacrimal duct 1 is left unfixed, the tip is unsettled to stimulate the conjunctiva, to thereby cause inflammation. Therefore, it is necessary to fix the tip. Lastly, the small holes A, B and C provided at three points in the skin are sutured, to thereby complete the placement operation of the artificial lacrimal duct. The above operation is very simple, and the required time from the skin incision to the suture of the incised part is about 30 to 50 minutes for one eye. Even for both eyes, the operation will be completed within one and half hours. The sutured incised part will be adhered in about 10 days.

FIG. 4 is a diagram showing an example of use of a small pump in the method of treating eye diseases according to the present invention. The end of the artificial lacrimal duct 1 placed beneath the skin around the head and the neck is projected outside the skin at the rear part of the neck, and covered with a rubber cap as an end sealing means 3 at the exposed end portion. A small pump is connected to this rubber cap as a drug solution injection means 8, and mounted on the back at the rear of the neck of a cat, using an adaptic dressing 9.

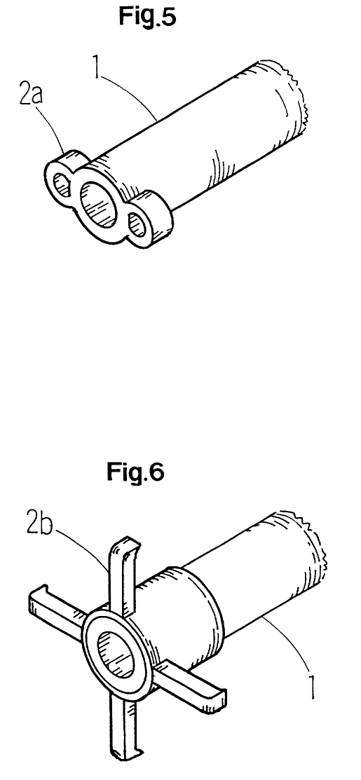

FIG. 5 is a perspective view showing an embodiment of a tip fixation means provided in the artificial lacrimal duct used in the present invention. A pair of ring-type small protrusions are integrally formed, as a tip fixation means 2a, with the artificial lacrimal duct, using the same material, at the tip portion of the artificial lacrimal duct 1. By suturing at two points using the two rings, fixation to the conjunctiva can be greatly strengthened.

FIG. 6 is a perspective view showing another embodiment of the tip fixation means provided in the artificial lacrimal duct used in the present invention. As a tip fixation means 2b, a cylinder having hook-type protrusions is separately formed, and this is fitted to the tip portion of the artificial lacrimal duct 1. By using this tip fixation means 2b, it is convenient to suture, because the time for suturing the tip of the artificial lacrimal duct 1 to the conjunctiva can be saved.

The method of treating eye diseases of animals constructed as described above was tested in an animal hospital operated by the present inventor, and it was confirmed that excellent effects more than expected was exhibited. That is to say, an artificial lacrimal duct was attached to the eyelid of a male dog (beagle, 3 years old) which received a large laceration in the cornea of the left eye, due to being scratched by a cat's claw. An electric pump with a timer for transfusion was connected to the end thereof to feed a mixture solution of an antibiotics and an artificial lacrimal fluid in an amount of 0.5 ml for every 3 to 4 hours every day and night. To our surprise, the laceration in the cornea, which had been expected to require 3 to 4 weeks until being completely cured with a conventional treatment by the instillation of drop, was adhered in only 10 days and was completely cured in 14 days. The experiments were also conducted with respect to five dogs and three cats which were infected with cataract or serious conjunctivitis. In each case, the animal made a satisfactory progress, and any problem was not observed at all from a clinical point of view.

Moreover, the experiments were evaluated very high from the owners of dogs and cats who took care of them at home after leaving the hospital, and it is no exaggeration to say that it won the highest praise. That is to say, they appreciated that not only they were released from the trouble for forcing application of eye drops or eyewashing to dogs and cats trying to avoid it by violently shaking their heads, but also the treatment of eye diseases could be easily and reliably performed. Particularly, the owner of a dog infected with keretoconjunctivitis sicca, which had to have the application of the artificial lacrimal fluid until its death, was pleased with that by making the dog carry a small pump with a timer, it became not necessary for him to wake up in the middle of night for applying eye drops.

If pressed to mention its defect, since the hair around the head and the neck is completely shaved at the time of placement operation of the artificial lacrimal duct, the appearance of dogs or cats after the operation is damaged. However, the hair will grow to the original state in about two months. So, if the owner has agreed with it beforehand, there is no problem.

As is obvious from the above description, with the method of treating eye diseases of the present invention, an artificial lacrimal duct opened in the conjunctiva on the backside of the eyelid is placed beneath the skin around the head and the neck, and a drug solution injection means such as a syringe or a small pump is fitted to the end thereof opened within the skin or outside the skin at the rear of the neck. As a result, the drug solution such as an eye disease remedium, eyewash or an artificial lacrimal fluid can be easily and reliably forced out towards the conjunctiva in the eyelid or the surface of the eyeball. Hence, it is not necessary to force open the eyelid of an animal trying to avoid application of eye drops or eyewashing to forcibly perform the treatment, thereby enabling alleviation of a burden imposed on the veterinarians and owners of animals.

Furthermore, it has been heretofore quite difficult to perform application of eye drops or washing eyes in the middle of night. With the method of treating eye diseases of the present invention, however, by connecting a small pump with a timer at the end of an artificial lacrimal duct, it is possible to pour the drug solution automatically to the diseased part for every several hours, all day long including the nighttime. Hence, an ideal treatment becomes possible, enabling a great reduction of the period required for the treatment of eye diseases.

The tip of the artificial lacrimal duct used in the method of treating eye diseases of the present invention can be reliably fixed to the conjunctiva on the backside of the eyelid, by providing a tip fixation means such as a ring or a hook at the tip portion thereof Moreover, when the artificial lacrimal duct is inserted to beneath the skin using an alligator forceps, since the tip fixation means protrudes, it can be reliably clamped by the alligator at the tip of the forceps.

What is claimed is:

1. A method of treating eye diseases of animals, wherein a drug solution is injected to the end of an artificial lacrimal duct placed beneath the skin of the head and the neck from the eyelid toward the rear part of the neck, so that the drug solution can be released from the tip of the artificial lacrimal duct opened in the conjunctiva on the backside of the eyelid.

2. An artificial lacrimal duct used in the method according to claim 1, wherein a tip fixation means is provided in an appropriate shape such as a ring for suturing the tip of the artificial lacrimal duct to the conjunctiva, or a hook for hooking it to the conjunctiva, at the tip portion of a soft long and slender tube.
